(19) United States
(12) Patent Application Publication
(10) Pub. No.: US 2019/0126267 A1
(43) Pub. Date: May 2, 2019

(54) DRIED BIOLOGICAL FLUID SPOT DEVICE

(71) Applicants: Issam Kabbani, Torrance, CA (US); Chenggong Zong, Los Angeles, CA (US)

(72) Inventors: Issam Kabbani, Torrance, CA (US); Chenggong Zong, Los Angeles, CA (US)

(21) Appl. No.: 16/179,129

(22) Filed: Nov. 2, 2018

Related U.S. Application Data
(60) Provisional application No. 62/580,828, filed on Nov. 2, 2017.

(51) Int. Cl. B01L 3/00; G01N 1/28

(52) U.S. Cl. CPC B01L 3/5023; G01N 33/49; G01N 1/286

(57) ABSTRACT

Described is a dried biological fluid spot device that is formed in a cassette-like shape for easy storage and transportation. The spot device includes a top portion having one or more openings passing therethrough and a bottom portion formed to affix with the top portion. Within the spot device is a pad holder that is formed to securely hold and suspend an absorbance pad such that the absorbance pad is positioned proximate the opening. Thus, during use, an operator can deposit a biological fluid through the opening and into the absorbance pad, where it is securely stored for transport and processing.

### CROSS-REFERENCE TO RELATED APPLICATIONS

The present application is a Non-Provisional Utility patent application of U.S. Provisional Application No. 62/580,828, filed on Nov. 2, 2017.

### BACKGROUND OF THE INVENTION

### (1) Field of Invention

The present invention relates to collection and storage of biological fluids and, more particularly, to a dried biological fluid spot device for collecting, drying, storing and shipping biologically relevant fluid.

### (2) Description of Related Art

A dried biological-fluid spot (DBS) device is an important tool of diagnostic and pharmacodynamic applications. Historically, DBS has been primarily used in the area of new born screening for inborn genetic disorder of infants. In this particular application, capillary blood is collected from the heel of infants, spotted onto a piece of filter paper and dried for future test. Relevant chemicals are significantly more stable in DBS than its liquid form, which permits longer shelf life and easier transport protocol (ambient temperature, smaller size). Upon analysis, a portion of dried blood on the filter paper is excised with a hole-puncher. Results obtained from this portion of dried blood are then used to differentiate infants with diseases from healthy ones.

Recently, the utilities of DBS in pharmacodynamics studies are growing rapidly, especially after the Food and Drug Administration (FDA) approved the usage of DBS-based assays for studies of drug-development and clinical trials. Meanwhile, the utilities of DBS in clinical diagnostics and health monitoring are emerging. A few variances of DBS device are used for these applications. Traditional filter paper, filter paper machined to a certain shape and size, and polymeric material machined to a certain shape and size co-exist in the market. To achieve a quantitative loading, the hypothesis is that either a quantitative amount of dried blood can be pouched out of a filter paper reproducibly, or quantitative amount of blood can be absorbed onto absorbent materials of specific sizes and shapes.

However, current DBS devices have shortcomings in fully accommodating the needs of these new assays. Traditional filter paper-based DBS device requires punching the filter paper with a hole-puncher. Variations in blood spotting (overloading, under-loading, irregular shape of dried spots) and sampling cause variations in amount of specimen used in an assay, thus limiting its reliability in quantitative assays. Moreover, filter paper is of limited mechanical strength, which renders it prone to disintegrate during the process of extracting chemicals from the DBS. Filter paper may also embody interference substances, which leach out during the extraction process affection assay results. As new DBS devices with polymeric materials are rolling out, many limitations persist. Loading of biological fluid to these devices are through direct contact of biological fluid of the absorbance material. Thus, the speed of sample loading and consistency of sample loading depends on many parameters. Under-loading and overloading both exist, which pose significant challenges to sampling accuracy and reproducibility. Therefore, all quantitative assays with the presumption that each DBS device is loaded with equal volume of biological fluid would be affected by this variance. With non-professional trained users (e.g., home users), this variance would trend bigger and less predictable. Moreover, a direct contact absorption mechanism requires the size of the collection device to be fairly-large. One end would be coated with absorbent materials while the other end serves as a hand-hold. This leads to two drawbacks. A large collection device is more difficult to fit into a vessel during the subsequent extraction procedure; especially considering that the volume of the extraction solvent is in the micro-liter range. Additionally, by holding the other end of the DBS device, the operator's hands may introduce interference substances. This is especially true with a non-professional user in a household situation, where many variety of interference substances exist.

Therefore, a continuing need exists for a new DBS device that addresses these challenges, which would pave the foundation for an array of precision quantitative assays.

### SUMMARY OF INVENTION

This disclosure provides a dried biological fluid spot device that is formed to provide for easy storage and transportation of a biological fluid. The spot device includes a top portion having one or more openings passing therethrough and a bottom portion formed to affix with the top portion. Within the spot device is a pad holder that is formed to securely hold and suspend an absorbance pad such that the absorbance pad is positioned proximate the opening. Thus, during use, an operator can use a capillary to easily deposit a biological fluid through the opening and into the absorbance pad, where it is securely stored for transport and processing.

In some aspects, the spot device includes an absorbance pad held by the pad holder and secured within the spot device.

In yet other aspects, a pad fixture is attached with the bottom portion. Further, the pad holder rises from the bottom portion to matingly engage with the pad fixture such that a peripheral portion of the absorbance pad is securely affixed between the pad holder and pad fixture, while a central portion of the absorbance pad is suspended and free from contact with top and bottom portions.

In yet another aspect, a desiccant is affixed within the spot device.

Further, the absorbance pad is formed to include of least one of PolyAmide (PA) and PolyVinyl Alcohol (PVA) (or both).

Finally, as can be appreciated by one in the art, the present invention also comprises a method for forming and using the invention described herein.

### BRIEF DESCRIPTION OF THE DRAWINGS

The objects, features and advantages of the present invention will be apparent from the following detailed descriptions of the various aspects of the invention in conjunction with reference to the following drawings, where:

FIG. 1 is a bottom, isometric-view illustration of a dried biological fluid spot device according to various embodiments of the present invention;

FIG. 2 is a top, isometric-view illustration of the dried biological fluid spot device;

FIG. 3 is a top, isometric, exploded-view illustration of the dried biological fluid spot device; and

FIG. 4 is a bottom, isometric, exploded-view illustration of the dried biological fluid spot device;

FIG. 5 is a cross-sectional, side, close-up view illustration of an absorbance pad being held in place by a pad holder and pad fixture according to various embodiments of the present invention;

FIG. 6 is a top-view illustration of the dried biological fluid spot device; and

FIG. 7 is a cross-sectional, side-view illustration of the dried biological fluid spot device, taken from line A-A of FIG. 5.

### DETAILED DESCRIPTION

The present invention relates to collection and storage of biological fluids and, more particularly, to a dried biological fluid spot device for collecting, drying, storing and shipping biologically relevant fluid. The following description is presented to enable one of ordinary skill in the art to make and use the invention and to incorporate it in the context of particular applications. Various modifications, as well as a variety of uses in different applications will be readily apparent to those skilled in the art, and the general principles defined herein may be applied to a wide range of embodiments. Thus, the present invention is not intended to be limited to the embodiments presented, but is to be accorded the widest scope consistent with the principles and novel features disclosed herein.

In the following detailed description, numerous specific details are set forth in order to provide a more thorough understanding of the present invention. However, it will be apparent to one skilled in the art that the present invention may be practiced without necessarily being limited to these specific details. In other instances, well-known structures and devices are shown in block diagram form, rather than in detail, in order to avoid obscuring the present invention.

The reader's attention is directed to all papers and documents which are filed concurrently with this specification and which are open to public inspection with this specification, and the contents of all such papers and documents are incorporated herein by reference. All the features disclosed in this specification, (including any accompanying claims, abstract, and drawings) may be replaced by alternative features serving the same, equivalent or similar purpose, unless expressly stated otherwise. Thus, unless expressly stated otherwise, each feature disclosed is only one example of a generic series of equivalent or similar features.

Furthermore, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of” or “act of” in the claims herein is not intended to invoke the provisions of 35 U.S.C. 112, Paragraph 6.

Please note, if used, the labels left, right, front, back, top, bottom, forward, reverse, clockwise and counter clockwise have been used for convenience purposes only and are not intended to imply any particular fixed direction. Instead, they are used to reflect relative locations and/or directions between various portions of an object.

(1) Introduction

Biological fluids carry specific molecular information on pathophysiological and pharmacodynamic state of people and animals. The device of this invention allows for the collection of such material. Specifically, this disclosure is directed to a dried biological fluid spot device for collecting, drying, protecting, shipping and storing biologically relevant fluid collection from Human or animal origins. Relevant biological fluids include but are not limited to blood (e.g., whole blood, serum, plasma, etc), urine, oral fluid (i.e., saliva), CSF (i.e., cerebrospinal fluid), amniotic fluid, synovial fluid, lymphatic fluid, breast milk, semen, sweat, peritoneal fluid, and tears, etc. These dried fluids carry characteristic chemicals and elements indicative of biologic/pathophysiologic/pharmacodynamic information of human or animals, which can be used in diagnostics, health monitoring, as well as research and development.

The design, engineering and production of the device of the present disclosure ensures precise collection of a defined volume of these fluids via the combination of highly-absorbent polymeric materials and measuring capillary. This device also ensures fast and efficient drying procedure. Further, the device provides protection to mechanical impacts, ambient moisture and environmental contaminants. In various embodiments, a unique identifier is given to each device in the form of both character combinations and/or a code (e.g., QR code) for the purpose of inventory and quick reading, respectively. Further details are provided below.

(2) Specific Details

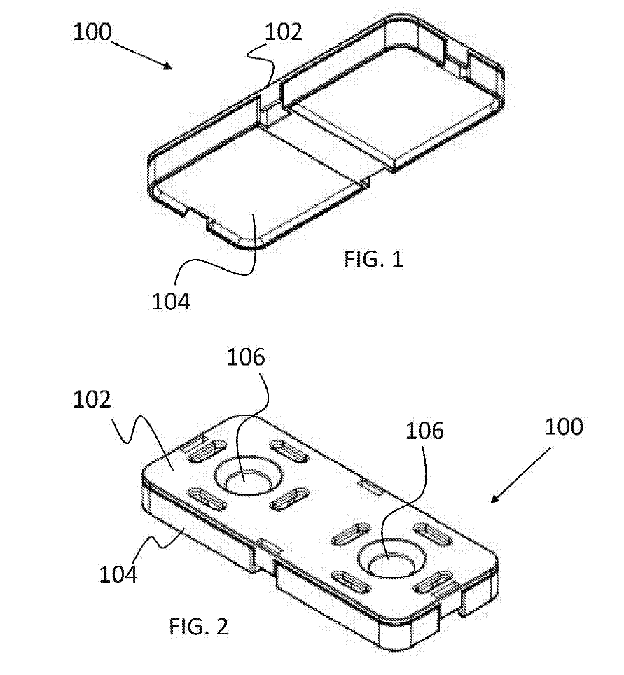

As noted above and as illustrated in FIGS. 1 through 7, the present disclosure is directed to a dried biological fluid spot device 100. FIGS. 1 and 2 illustrate bottom and top views, respectively, of the spot device 100. The spot device 100 operates as an enclosure to securely hold one or more pads for absorbing biological fluids. In various embodiments, the spot device 100 includes a cover portion 102 connected with a base portion 104 (which collectively form the enclosure). As shown in FIG. 2, the cover portion 102 includes one or more openings 106 that pass through the cover portion 102 and into the enclosure.

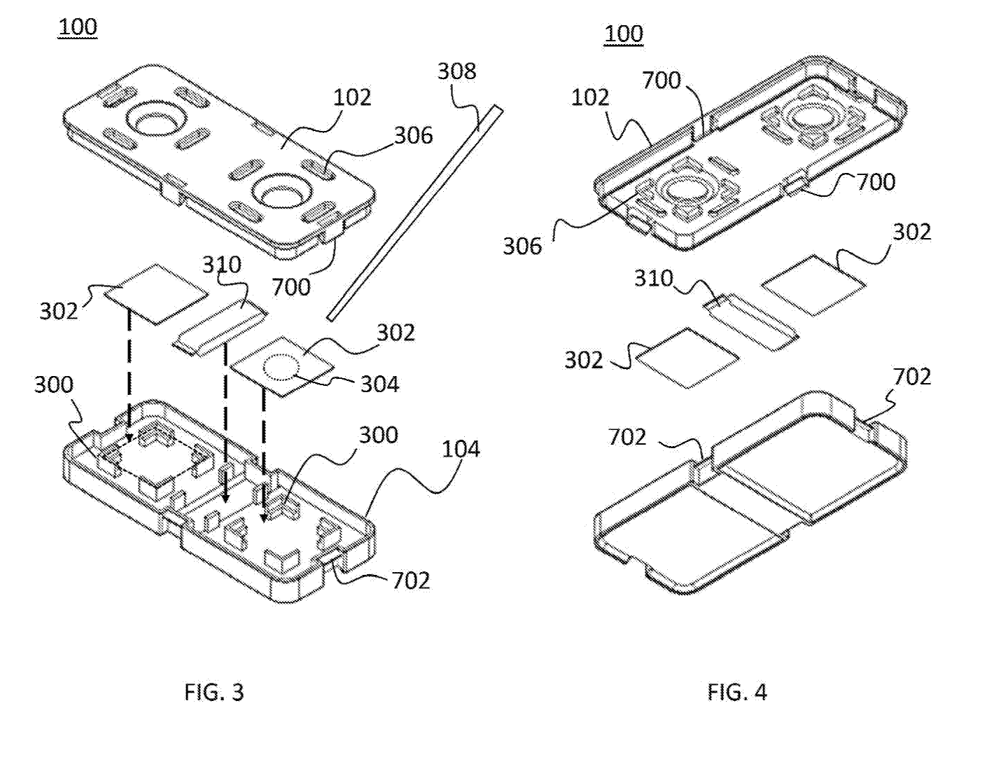

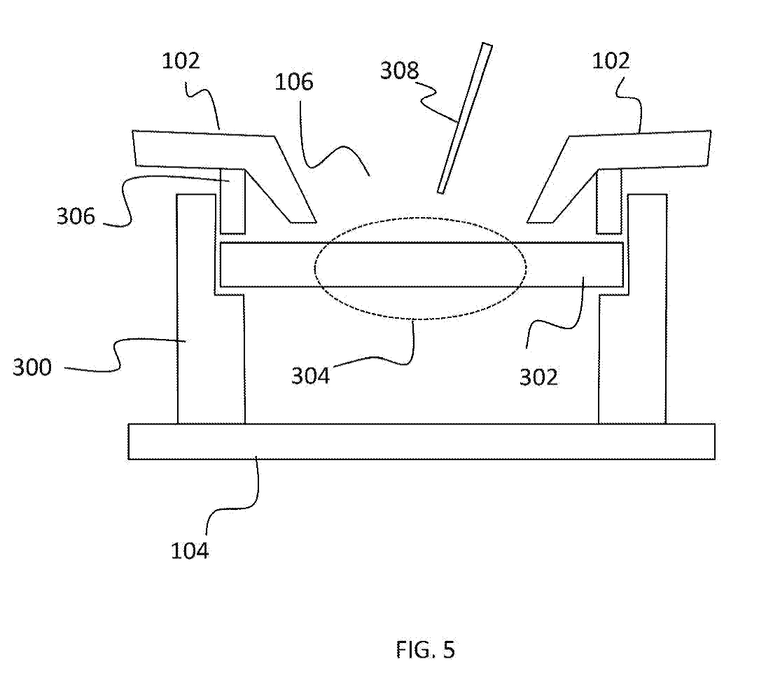

FIGS. 3 and 4 provide exploded view illustrations of the spot device 100. As shown, the bottom portion 104 includes one or more raised pad holders 300 that are formed to securely hold an absorbance pad 302. The pad holders 300 are formed in any suitable shape such that they can hold a corresponding pad 302 in a suspended position such that at least a center portion 304 of the pad remains free from touching any surfaces of the spot device 100. As a non-limiting example, the pad holders 300 are raised ledges that are shaped to correspond to the shape of the pad 302. Similarly, the top portion 102 can desirably include pad fixtures 306 that are formed to securely affix the pads 302 with the pad holders 300. For example, when a pad 300 is positioned within the pad holder 300, the top portion 102 can be pressed onto the bottom portion 104, which forces the pad fixtures 306 into a locking engagement with the pad holders 300 to securely suspend the pad 300 between the top portion 102 and bottom portion 104. This is further illustrated in the close-up, side-view illustration as provided in FIG. 5.

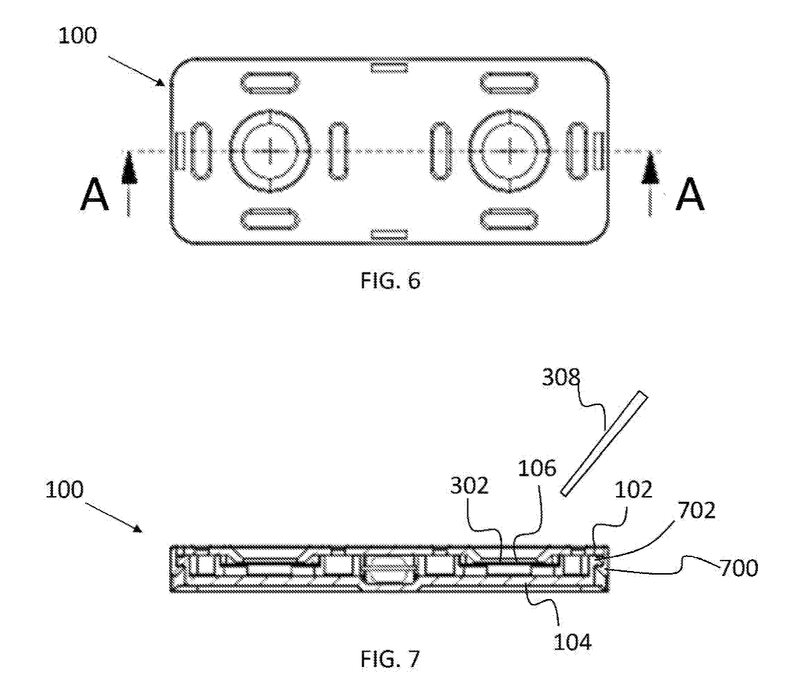

As shown in FIG. 5, when the top portion 102 is positioned onto the bottom portion 104, the pad 300 is securely held in a suspended positioned by being clamped between the pad holder 302 and corresponding pad fixture 306. Notably and as referenced above, the central portion 304 of the pad is both suspended within the spot device 100 and positioned beneath an opening 106 in the cover portion 102. This aspect is further illustrated in FIG. 7, which provides a cross-sectional, side-view illustration of the spot device 100 as taken from line A-A of FIG. 6. As shown in FIG. 7, the pad 302 is suspended and held securely within the spot device 100 and beneath the opening 106 of the top portion 102. The opening 106 provides a hole through the top portion 102 with ample space to accommodate a capillary 308 to dispense the biological fluid from the capillary 308 onto the pad 302. With biological fluid positioned upon the pad 302, the spot device 100 can be deposited or otherwise secured within a secondary enclosure for storage and transport. In some aspects, the secondary enclosure is simply an envelope or bag, while in other aspects, the secondary enclosure can be formed as a case that or shell that securely seals the spot device 100 therein. In some aspects, the secondary enclosure is air tight to prevent any further moisture or contaminants from entering the spot device 100.

It should be noted that the spot device 100 can include a locking device to lock the spot device 100 and secure the pad 302 within the spot device 100. As can be appreciated, there are several techniques by which the spot device 100 can be locked. As a non-limiting example, the locking device is formed such that the top portion 102 includes top lock portion 700 that locks against a bottom lock portion 702 formed on the bottom portion 104. For example and as further illustrated in FIGS. 3 and 4, the top lock portion 700 can be formed to include a latch that locks against a bottom lock portion 702 (which can be formed as a corresponding catch). Thus, in this example, the locking device includes a latch and catch. Thus, the latch (top lock portion 700) simply locks against a corresponding catch (bottom lock portion 702) to securely affix the top portion 102 against the bottom portion 104. In doing so, the pad 302 is securely affixed within the enclosure or spot device 100. In a clinical laboratory or other setting, these locks can be opened with a specialized tool; thus, the pad 302 can be released from the spot device 100 without mechanical stress being applied directly to the pad 302. For example, a tool can be used to simply pry the top lock portion 700 (latch) off of the bottom lock portion 702 (catch) to allow a user to lift the top portion 102 from the bottom portion 104 to expose the pads 302 for withdrawal and processing.

Referring again to FIGS. 3 and 4, the spot device 100 also desirably includes a desiccant 310 or desiccant pack. The spot device 100 is formed to securely hold the desiccant 310 within the enclosure to efficiently extract moisture from the pad 302, spot device 100, and/or biological fluids.

As shown in FIGS. 3 and 5, the spot device 100 can include a corresponding calibrated measuring capillary 308 that can be used to collect biological fluid and position the biological fluid onto the pads 302 through the opening 106. The accurate volume measurement of biological fluid is ensured with the calibrated measuring capillary 308. The capillary can be pre-loaded with heparin (or other relevant material) to prevent clotting. When pre-loaded with an anticoagulant or other material, the accuracy of volume measurement is not comprised during its usage because the capillary 308 can be precisely sized to accommodate the anticoagulant or other drawn in fluid. Thus, given the capillary 308, an accurate volume of biological fluids (20 μL or 40 μL, etc.) can be loaded and unloaded from the capillary 308 (i.e., capillary tube).

Further, with the measuring capillary 308, the volume accuracy of collected biological fluid is not affected by the size of the pad 302 as long as its absorbing capacity exceeds the capacity of the capillary 308. A slightly larger pad 302 aids and shortens the drying process. As a non-limiting example, the size of a 20 μL or 40 μL pad 302 is suited to be loaded into a standard 1.5 mL Eppendorf tube or deep-well microplate for subsequent extraction process.

The pads 300 are made of any suitable highly-absorbent material and are desirably pre-cut to align with and securely connect with the shaping or configuration as framed or otherwise formed by the pad holders 302. Desirably, the pads are made of a highly-absorbent materials such as PolyAmide (PA) and/or PolyVinyl Alcohol (PVA).

Such PA and PVA based polymeric materials are highly absorbent, while they do not leach out interference substances during extraction. Further and to be contrasted with the prior art, these materials are non-frit and do not tear or disintegrate, which would not release lint or fibers. Moreover, the mechanical strength of these materials allows for the pads 302 to be suspended due to their rigidity to avoid contamination. Further, the mechanical strength allows the pads 302 to tolerate vigorous vortexing, sonication and beads-beating, which may be used in a laboratory for extraction of the biological samples held by the pads 302.

In summary, this disclosure provides a spot device 100 with one or more absorbance pads. Each absorbance pad 302 is made of non-streak, non-fraying, non-lint material, which enables it to maintain structural integrity under harsh extraction conditions, including but not limited to sonication, bead-beating, vortexing, extended soaking in aqueous solution as well as certain organic solvents (i.e., methanol). The flat shape of the pad 302 supports even extraction across the whole pad. Further, the shape of the pad 302 makes it compatible with existing automated Dried Blood System extraction apparatus on the market. With a fixed volume capillary or pipette, the device is suitable for both quantitative and qualitative sample collection. Additionally and in some aspects, the cassette configuration/shape (as shown in the figures) of the spot device 100 enables easy disassembly of the protective enclosure (e.g., top portion 102 and bottom portion 104) and easy retrieval of the absorbance pad 302. Further, the cassette configuration enables future utility to be developed to collect serum and plasma.

Finally, while this invention has been described in terms of several embodiments, one of ordinary skill in the art will readily recognize that the invention may have other applications in other environments. It should be noted that many embodiments and implementations are possible. Further, the following claims are in no way intended to limit the scope of the present invention to the specific embodiments described above. In addition, any recitation of “means for” is intended to evoke a means-plus-function reading of an element and a claim, whereas, any elements that do not specifically use the recitation “means for”, are not intended to be read as means-plus-function elements, even if the claim otherwise includes the word “means”. Further, while particular method steps have been recited in a particular order, the method steps may occur in any desired order and fall within the scope of the present invention.

What is claimed is:

1. A dried biological fluid spot device, comprising:
a top portion having one or more openings passing therethrough;
a bottom portion formed to affix with the top portion; and
a pad holder formed to securely hold and suspend an absorbance pad within the spot device such that the absorbance pad is positioned proximate the opening.

2. The dried biological fluid spot device as set forth in claim 1, further comprising an absorbance pad held by the pad holder and secured within the spot device.

3. The dried biological fluid spot device as set forth in claim 2, further comprising a pad fixture attached with the bottom portion, and wherein the pad holder rises from the bottom portion to matingly engage with the pad fixture such that a peripheral portion of the absorbance pad is securely affixed between the pad holder and pad fixture, while a central portion of the absorbance pad is suspended and free from contact with top and bottom portions.

4. The dried biological fluid spot device as set forth in claim 3, further comprising a desiccant affixed within the spot device.

5. The dried biological fluid spot device as set forth in claim 4, wherein the absorbance pad is formed to include of least one of PolyAmide (PA) and PolyVinyl Alcohol (PVA).

6. The dried biological fluid spot device as set forth in claim 2, wherein the absorbance pad is formed to include at least one of PolyAmide (PA) and PolyVinyl Alcohol (PVA).

7. The dried biological fluid spot device as set forth in claim 1, further comprising a desiccant affixed within the spot device.
